(19) United States
(12) Patent Application Publication
(10) Pub. No.: US 2011/0160727 A1
(43) Pub. Date: Jun. 30, 2011

(54) RIGID SKULL FIXATION

(75) Inventor: Thomas ARN, Lidingo (SE)

(73) Assignee: ELEKTA AB (PUBL), Stockholm (SE)

(21) Appl. No.: 12/650,167

(22) Filed: Dec. 30, 2009

(51) Int. Cl. A61B 17/00

(52) U.S. Cl. 606/59

(57) ABSTRACT

The invention relates to a stabilizing device adapted to be applied to a stereotactic frame (2, 3), which is provided with fixation pins (4) to allow fixation of the stereotactic frame to a bone (5) of a human or animal body part to be examined or treated, wherein the stabilizing device is adapted to stabilize a fixation pin in relation to the frame. The stabilizing device comprises a stabilizing member (9) which in a first position is provided with a through opening (13), adapted to be applied over the fixation pin in an area between the frame and the body part, and a tensioning member (10) positioned in a second position on a distance from the through opening and being capable of drawing the stabilizing member towards the frame. The stabilizing member is formed with a stop member (12), which is adapted, when tightening the tensioning member, to prevent displacement of the first position of the stabilizing member along the fixation pin towards the frame, while allowing displacement of the second position of the stabilizing member towards the frame, such that the fixation pin will be drawn or bent in a direction towards the second position.

The present invention relates to a stabilizing device adapted to be applied to a stereotactic frame, which is provided with fixation pins to allow fixation of the stereotactic frame to a bone of a human or animal body part to be examined or treated, wherein the stabilizing device is adapted to stabilize a fixation pin in relation to the frame.

### BACKGROUND OF THE INVENTION

A stereotactic frame is used for establishing a reference system to localize areas to be treated during imaging such as MRI-scanning of a head of a patient and/or for supporting instruments during neurological surgery, therapy or diagnosis. The stereotactic frame is arranged around the head and fixation pins connected to the frame are screwed into or to abutment against the bone of the skull to ensure a rigid fixation of the reference system.

However, a small play is always present between each fixation pin and the frame even if it is tried to obtain a tight fit. Such a play will make the attachment to the skull unstable and will have an especially large influence on the position of the frame in relation to the head in a case where the distance from the frame to the skull is very long, due to for example fixation to a small skull, such as a skull of a child where very long fixation pins have to be used. In some cases, such as is disclosed in WO 03/094769 and WO 05/104975, auxiliary sleeve members are used to mount the fixation pins. The auxiliary sleeve members have to result that the play will become even larger since a play will appear both between the frame and the sleeve member as well as between the sleeve member and the fixation pin. Moreover, in WO 03/094769 the sleeve members are formed of a plastic material, in order to reduce heat generated during MRI-scanning, which usually will increase the play further in relation to attachment of the fixation pins in a metallic material.

### SUMMARY OF THE INVENTION

It is an object of the invention to provide a stabilizing device, which is simple and inexpensive to manufacture and which in a reliable way is capable of stabilizing a fixation pin in relation to a stereotactic frame. At least this object is achieved by a stabilizing device according to claim 1.

Accordingly, the basis of the invention is the insight that the above object may be achieved by providing a stabilizing member, which is provided with a through opening adapted to be applied over the fixation pin in an area between the frame and the body part. On a distance from the through opening, the stabilizing member is provided with a tensioning member, which is capable to draw the stabilizing member towards the frame. Furthermore, the stabilizing device is also provided with a stop member, which is adapted to prevent displacement of the portion at the through opening along the fixation pin towards the frame when tightening the tensioning member, while at the same time the portion at the tensioning member is displaced towards the frame. This has to result that the stabilizing member will be drawn in an obliquely way in relation to the fixation pin and the frame such that the fixation pin is drawn or bent in a direction towards the tensioning member and by that the play between the fixation pin and the frame will be eliminated.

Within this overall idea, the invention can be realized and modified in many different ways within the scope of the claims. As an example, the tensioning member is in all of the hereinafter described and illustrated embodiments in form of a screw having an external thread, which can be engaged in a hole having an internal thread in the stabilizing member. However, it is to be understood that the tensioning member could be formed also in many other ways, e.g. as a flexible member which can be tightened between the stabilizing member and the frame.

In case the tensioning member is in form of a rigid member, e.g. as a threaded screw, it is preferred that the hole in the frame is in form of an elongated slot, since during tightening of the screw and subsequent displacement of the portion of the stabilizing member at the tensioning member, the angle between the screw hole in the stabilizing member and the frame will be slightly altered. With the screw inserted through an elongated slot, this alteration of the angle can be met without, or at least with a reduced, bending of the screw by a slightly displacement of the screw along the elongated slot.

Moreover, the through opening in the stabilizing member for the fixation pin can be shaped in different ways. In one embodiment described and illustrated hereinafter, the opening is illustrated as a recess, which is open at one side of the stabilizing member and has a hook formed formation in a portion being located on an opposite side of the opening in relation to the tensioning member. In this way the mounting of the stabilizing member onto the fixation pin is facilitated and even allows mounting of the stabilizing member after the frame has been attached to a skull. In another embodiment the through opening is formed as a hole having a diameter slightly larger than the diameter of the fixation pin. Such an embodiment has to be applied on the fixation pin before attaching the frame to a skull.

An important feature of the invention is that the stabilizing member is formed with some kind of stop member which, when the stabilizing member is mounted onto the fixation pin of the frame and the tensioning member is tightened, will prevent displacement of the stabilizing member at a first position, at the through opening for the fixation pin, along the fixation pin in a direction towards the frame, while at the same time a displacement of a second position, at the tensioning member, towards the frame is allowed. Such a stop member can be realized in many different ways and examples will be described more in detail in connection with hereinafter described and illustrated embodiments of the invention. A common feature with all of the embodiments is that when tightening the tensioning member, the first position of the stabilizing member will be slightly drawn towards the frame such that the stabilizing member including the through opening around the fixation pin, will be inclined in relation to the fixation pin such that the fixation pin will be drawn and/or bent towards the tensioning member which will eliminate any play between the fixation pin and the frame.

It is to be understood that the invention can be applied on many different kinds of stereotactic frames. In a hereinafter described and illustrated embodiment, the frame comprises an annular support structure, which carries four upward directed posts, which each is provided with a fixation pin in its upper end. However, the support structure does not have to be annular, but could also be e.g. U-shaped, and does not have to be provided with posts. Instead, the fixation pins could be provided directly in the support structure. Furthermore, in the described and illustrated embodiments, the stabilizing member is mounted such that the tensioning member is located beneath the fixation pin. Naturally, the tensioning member could be located above the fixation pin and, for example in case the fixation pins are attached directly in the support structure, also laterally in relation to the fixation pin. The stabilizing members according to the hereinafter described and illustrated embodiments are shown mounted to a stereotactic frame in which the fixation pins are mounted in an auxiliary sleeve member, e.g. of an electrically insulating material for restricting the electrical coupling between the fixation pin and the post to reduce heating during MRI imaging. It is to be understood, however, that the inventive stabilizing member also could be mounted to a stereotactic frame in which the fixation pins are attached directly into the frame. Moreover, the stereotactic frame is illustrated as being attached to the head of a patient. However, a similar stereotactic frame could also be attached to other parts of a human or animal body.

### BRIEF DESCRIPTION OF THE DRAWINGS

Embodiments of the invention will hereinafter be described by way of example with reference to the accompanying drawings, in which:

FIG. 1 is a perspective view of a stereotactic frame being attached to a human head;

FIG. 2 is a schematic, partly cut through side view of a part of a stereotactic frame being attached to a head and being provided with a stabilizing device according to a first embodiment of the invention;

FIG. 3 is a perspective view of a stabilizing member according to an embodiment being provided with a through opening formed as a recess;

FIG. 4 is a perspective view of a stabilizing member according to an embodiment being provided with a through opening formed as a hole; and

FIGS. 5-9 are side views of a post of a stereotactic frame being provided with stabilizing devices of alternative embodiments according to the invention.

### DETAILED DESCRIPTION OF EMBODIMENTS OF THE INVENTION

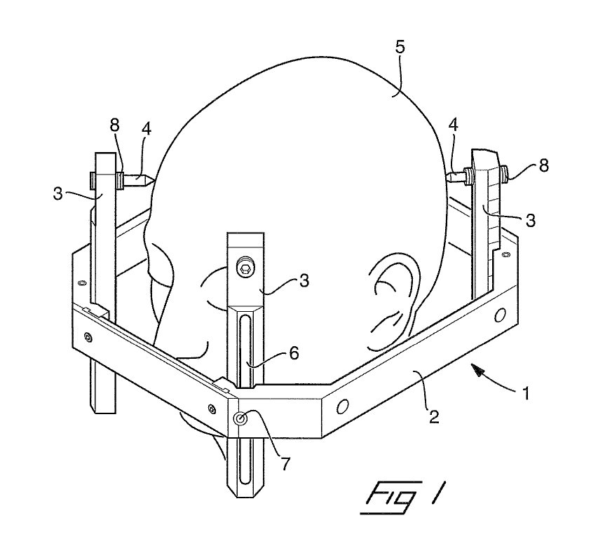

Reference is first made to FIG. 1 in which is illustrated a stereotactic frame 1 of a type being well known in prior art. The stereotactic frame comprises an annular support structure 2, having a generally rectangular shape, which in each corner section is provided with upward projecting posts 3. A fixation pin 4 is arranged in the upper end of each post and screwed with a front sharp point into the skull 5 of a patient such that the stereotactic frame is fixated to the skull. In order to adopt the stereotactic frame to different conditions, each post is provided with an elongated through slot 6 and attached to the support structure by means of a screw 7 or the like extending through the slot, such that each post can be regulated in height. Such a stereotactic frame can be used as a reference system when viewing the head by means of e.g. MRI scanning or treatment by means of x-ray radiation, and as a support for instruments or the like e.g. when taking of biopsies or performing surgery. Accordingly, it is important that the stereotactic frame will be secure fixed to the skull, i.e. that the fixation pins are secure attached to the skull as well as to the posts, such that the risk of taking samples of or treating a healthy area of the brain is eliminated. The illustrated stereotactic frame is adapted to reduce heat generated during MRI scanning due to electrical coupling between the posts and the fixation pins. For this reason, each fixation pin is accommodated in an auxiliary sleeve 8, of an electrically insulating material, which is positioned in a through hole in each post. Such auxiliary sleeves will contribute to an increased play between each fixation pin and post.

Reference is then made to FIG. 2 in which is illustrated a part of the stereotactic frame, including the annular support structure 2 and the post 3, and a fixation pin 4 being screwed into the skull 5 of a patient, and a stabilizing device, according to a first embodiment of the invention, which is mounted to stabilize the fixation pin in relation to the post. The stabilizing device comprises a stabilizing member 9 and a tensioning member 10 in form of a screw having an external thread. The stabilizing member 9 is in form of a rigid body comprising an upright portion 11 and a laterally directed projection 12 in the lower end portion of the stabilizing member. A through opening 13 for the fixation pin 4 is formed in the upper end portion of the stabilizing member and a hole 14 having an internal thread for the tensioning screw is formed in the projection 12. An end surface 15 of the projection, which in the mounted state, as shown in FIG. 2, is adapted to bear against the post 3, is inclined and presents an angle in relation to a plane perpendicular to the threaded hole and parallel to the post, such that the projection is shorter in the lower end of the stabilizing member, i.e. in the end of the stabilizing member being located furthest away from the through opening 13, than at the junction of the projection 12 and the upright portion 11. Accordingly, when positioning the stabilizing member with the through opening 13 over the fixation pin 4 in an area between the post 3 and the skull 5, as illustrated in FIG. 2, and the tensioning screw 10 is passed through the slot 6 (FIG. 1) in the post and screwed into the threaded hole 14 in the projection 12 and subsequently tightened, the projection will function as a stop member which, due to the inclined end surface 15, prevents the upper end of the stabilizing member, i.e. the first position around the through opening 13 for the fixation pin, to be displaced towards the post 3, while the lower end of the stabilizing member, i.e. the second position around the threaded hole 14, will be displaced towards the post. In this way the upright portion 11 of the stabilizing member 9 will be drawn to a somewhat inclined position, as is illustrated in the drawing, such that the through opening 13 around the fixation pin will draw the fixation pin downwards and any play between the fixation pin and the post will be eliminated. Since the tensioning screw 10 is passed through the slot 6 in the post 3, the screw is free to be displaced somewhat upwards due to the angular displacement of the stabilizing member.

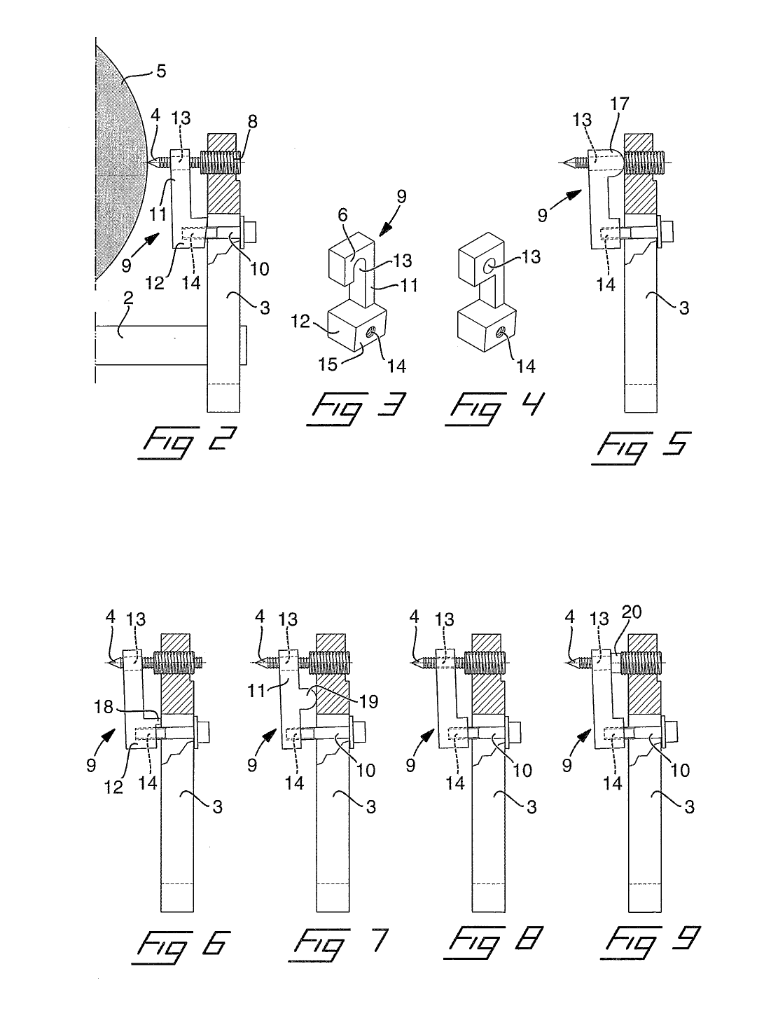

The opening through the stabilizing member for the fixation pin can be formed in different ways. In FIG. 3 is illustrated an embodiment where the through opening 13 is formed as a recess, which is open downwards/laterally and is formed with a hook 16 in an upper portion being located on an opposite side of the through opening in relation to the threaded hole 14 for the tensioning screw 10. Such an embodiment is advantageous in so far as the stabilizing member can be mounted afterwards when the stereotactic frame is already attached to the skull. An alternative embodiment is illustrated in FIG. 4 where the opening is formed as a through hole 13 having a diameter slightly larger than the diameter of the fixation pin 4. Such an embodiment of the stabilizing member must be positioned over the fixation pin prior to attaching the stereotactic frame to the skull.

FIG. 5 illustrates a second embodiment of the inventive stabilizing device. Here the stop member is formed as a projection 17 located at the area of the through opening 13 for the fixation pin and extending in the direction towards the post and has a rounded end surface. When tightening the tensioning screw, the projection will prevent displacement of the first position of the stabilizing member around the through opening for the fixation pin, while the second position of the stabilizing member around the threaded hole 14 for the tensioning screw will be drawn towards the post 3. In this way the fixation pin will be drawn and/or bent downwards such that the play between the fixation pin and the post will be eliminated.

A third embodiment of the stabilizing device, which is illustrated in FIG. 6, is similar to the first embodiment according to FIG. 2. However, here the end surface of the projection 12 is not inclined as in the first embodiment. Instead, the end surface is provided with a ridge in the upper portion of the projection which will function as a stop member that will prevent displacement of the first position of the stabilizing member but allow displacement of the second position when the tensioning screw is tightened.

A fourth embodiment of the stabilizing device is illustrated in FIG. 7. Here, the stabilizing member is in form of an upright portion 11 having a projection 19, at an intermediary position between the through opening 13 and the threaded hole 14 for the tensioning screw 10, which is directed towards the post 3 and has a rounded end surface. The projection 19 will function as a stop member which, when the tensioning screw is tightened, will prevent displacement of the first position around the through opening for the fixation pin towards the post, but will allow displacement of the second position around the threaded hole for the tensioning screw towards the post. Accordingly, the fixation pin 4 will be drawn/bent towards the tensioning screw eliminating any play between the fixation pin and the post.

FIG. 8 illustrates a fifth embodiment of the stabilizing device. In this embodiment the stabilizing member 9 is not formed with a stop member being adapted to bear against the post 3. Instead, the stop member is in form of a thread inside the through opening 13 for the fixation pin 4, which will interact with an external thread on the fixation pin. The interacting threads in the through opening and on the fixation pin will prevent displacement of the first position around the through opening towards the post, during tightening of the tensioning screw, while allowing displacement of the second position around the tensioning screw towards the post, which will have to effect that the fixation pin will be drawn/bent towards the tensioning screw, thus eliminating any play between the fixation pin and the post.

FIG. 9 illustrates a sixth embodiment of the stabilizing device. The stop member in this embodiment is formed by a sleeve member 20 located over the fixation pin 4 between the stabilizing member and the post. When tightening the tensioning screw 10, the sleeve member will prevent displacement of the first position around the through opening 13 towards the post, while the displacement of the second position around the threaded hole 14 towards the post is allowed. Accordingly, when tightening the tensioning screw the fixation pin will be drawn/bent towards the tensioning screw such that the play between the fixation pin and the post will be eliminated.

It is to be understood that the through opening 13 in the stabilizing member for the fixation pin optionally can be formed as in FIG. 3, i.e. as a recess being open downwards/laterally, or as in FIG. 4, i.e. as a hole, for all of the embodiments according to FIGS. 5-7 and 9. However, in the fifth embodiment according to FIG. 8 the through opening has to be formed as a threaded hole in order to allow engagement between the fixation pin 4 and the stabilizing member 9.

1. A stabilizing device adapted to be applied to a stereotactic frame, which is provided with fixation pins to allow fixation of the stereotactic frame to a bone of a human or animal body part to be examined or treated, wherein the stabilizing device is adapted to stabilize a fixation pin in relation to the frame,
the stabilizing device comprising a stabilizing member which in a first position is provided with a through opening, adapted to be applied over the fixation pin in an area between the frame and the body part, and a tensioning member positioned in a second position on a distance from the through opening and being capable of drawing the stabilizing member towards the frame,
wherein the stabilizing member is formed with a stop member, which is adapted, when tightening the tensioning member, to prevent displacement of the first position of the stabilizing member along the fixation pin towards the frame, while allowing displacement of the second position of the stabilizing member towards the frame, such that the fixation pin will be drawn or bent in a direction towards the second position.

2. A stabilizing device according to claim 1, wherein the tensioning member is a threaded screw, which is passed through an opening in the frame and screw threaded attached to the stabilizing member.

3. A stabilizing device according to claim 1, wherein the opening in the frame is an elongated slot.

4. A stabilizing device according to claim 1, wherein the through opening in the stabilizing member is a recess, which is laterally open and the stabilizing member is formed with a hook in a portion being located on an opposite side of the opening in relation to second position of the stabilizing member.

5. A stabilizing device according to claim 1, wherein the stabilizing member comprises a projection at the area of the second position which is projecting towards the frame and presents a contact surface adapted to bear against the frame.

6. A stabilizing device according to claim 5, wherein the contact surface is inclined in relation to a plane being perpendicular to the longitudinal direction of the tensioning member such that the projection is longer in a portion between the tensioning member and the through opening than on the opposite side of the tensioning member.

7. A stereotactic frame which is provided with fixation pins to allow fixation of the stereotactic frame to a bone of a human or animal body part to be examined or treated, comprising at least one stabilizing device according to any of the claim 1.

8. A stereotactic frame according to claim 7, wherein the stereotactic frame comprises an annular support structure and projecting posts, in which the fixation pins are attached.

9. A stabilizing device according to claim 2, wherein the opening in the frame is an elongated slot.

10. A stabilizing device according to claim 2, wherein the through opening in the stabilizing member is a recess, which is laterally open and the stabilizing member is formed with a hook in a portion being located on an opposite side of the opening in relation to second position of the stabilizing member.

11. A stabilizing device according to claim 3, wherein the through opening in the stabilizing member is a recess, which is laterally open and the stabilizing member is formed with a hook in a portion being located on an opposite side of the opening in relation to second position of the stabilizing member.

12. A stabilizing device according to claim 2, wherein the stabilizing member comprises a projection at the area of the second position which is projecting towards the frame and presents a contact surface adapted to bear against the frame.

13. A stabilizing device according to claim 3, wherein the stabilizing member comprises a projection at the area of the second position which is projecting towards the frame and presents a contact surface adapted to bear against the frame.

14. A stabilizing device according to claim 4, wherein the stabilizing member comprises a projection at the area of the second position which is projecting towards the frame and presents a contact surface adapted to bear against the frame.

15. A stereotactic frame which is provided with fixation pins to allow fixation of the stereotactic frame to a bone of a human or animal body part to be examined or treated, comprising at least one stabilizing device according to claim 2.

16. A stereotactic frame which is provided with fixation pins to allow fixation of the stereotactic frame to a bone of a human or animal body part to be examined or treated, comprising at least one stabilizing device according to claim 3.

17. A stereotactic frame which is provided with fixation pins to allow fixation of the stereotactic frame to a bone of a human or animal body part to be examined or treated, comprising at least one stabilizing device according to claim 4.

18. A stereotactic frame which is provided with fixation pins to allow fixation of the stereotactic frame to a bone of a human or animal body part to be examined or treated, comprising at least one stabilizing device according to claim 5.

19. A stereotactic frame which is provided with fixation pins to allow fixation of the stereotactic frame to a bone of a human or animal body part to be examined or treated, comprising at least one stabilizing device according to claim 6.
